(19) United States
(12) Patent Application Publication
(10) Pub. No.: US 2023/0272448 A1
(43) Pub. Date: Aug. 31, 2023

(54) AN IMPROVED SUBSTRATE COCKTAIL ASSAY FOR HIGH-THROUGHPUT SCREENING OF CYTOCHROME P450 INHIBITORS

(71) Applicant: PIRAMAL PHARMA LIMITED, Mumbai (IN)

(72) Inventors: Vinod SANNA, Ahmedabad (IN); Sharadsrikar KOTTURI, Ahmedabad (IN); Mahendra SHUKLA, Ahmedabad (IN); Mohammed Salauddin SHAIKH, Ahmedabad (IN); Himani SAMAR, Udaipur (IN)

(73) Assignee: PIRAMAL PHARMA LIMITED, Mumbai (IN)

(21) Appl. No.: 18/017,926

(22) PCT Filed: Jul. 30, 2021
(86) PCT No.: PCT/IB2021/056958

§ 371 Date: Jan. 25, 2023

(30) Foreign Application Priority Data
Jul. 31, 2020 (IN) 202021032923

(51) Int. Cl. C12Q 1/26

(52) U.S. Cl. CPC C12Q 1/26

(57) ABSTRACT

The present invention relates to obtaining single incubation condition for cocktail of probe substrates of 8 major Cytochrome P450 (CYP) isoforms to assess in vitro drug-drug interactions (DDIs) of novel drug candidates using liquid chromatography coupled to tandem mass spectrometry (LC-MS/MS) method. The present invention provides an improved and reduced substrate concentration and is developed in lower microsomal protein to avoid any non-specific binding of substrates with the microsomal proteins.

### FIELD OF THE INVENTION

The present invention relates to obtaining single incubation condition for cocktail of probe substrates of 8 major Cytochrome P450 (CYP) isoforms to assess in vitro drug-drug interactions (DDIs) of novel drug candidates using liquid chromatography coupled to tandem mass spectrometry (LC-MS/MS) method. The present invention provides an improved and reduced substrate concentration and is developed in lower microsomal protein to avoid any non-specific binding of substrates with the microsomal proteins.

### BACKGROUND OF THE INVENTION

The following discussion of the prior art is intended to present the invention in an appropriate technical context, and allows its significance to be properly appreciated. Unless clearly indicated to the contrary, reference to any prior art in this specification should not be construed as an expressed or implied admission that such art is widely known or forms part of common general knowledge in the field.

CYP enzymes are heme thiolate proteins that are responsible for the oxidative metabolism of a wide variety of xenobiotics. They comprise a superfamily of related enzymes that are grouped into families and subfamilies based on similarities in amino acid sequences. The five major human CYP enzymes responsible for the metabolism of xenobiotics are CYP1A2, CYP2C9, CYP2C19, CYP2D6, and CYP3A4. It is estimated that these five CYP enzymes are responsible for approximately 99% of CYP-mediated drug metabolism. The determination of the CYP enzymes responsible for the metabolism of new chemical entities (NCEs) and the identification of interactions with a specific CYP isozyme (e.g. inhibition of that isozyme) can aid in predicting clinical drug interactions.

During initial stages of drug development, assessing metabolic profile and potential drug-drug interactions (DDIs) in humans is mandatory. A novel drug candidate could be substrate, inhibitor or inducer of human metabolizing enzymes. Drug metabolism via the cytochrome P450 system has emerged as an important determinant in the occurrence of several DDIs that can result in toxicities, reduced pharmacological effect, and adverse drug reactions. It is the major cause of withdrawal in later stages of clinical trials and even from the market. To avoid this failure USFDA has recommended DDI assays for 7 major CYP450 isoforms: 3A4, 1A2, 2C9, 2C19, 2D6, 286, and 2C8. The traditional way to assess interaction individually with each CYP450 isoform has some drawbacks like being time consuming, labour intense and cost-ineffective. Subsequently, ‘n-in-one’ studies employing probe cocktail substrates have been reported as an alternative. However, choice of specific probe substrate for each isoform in cocktail remains a key factor because number of enzymes might be involved in the metabolism of a xenobiotic. To avoid interaction between probe substrates, USFDA has suggested highly specific CYP450 reaction markers, but the specificity is dependent on their concentrations.

PCT Publication No. WO02/04660 A2 discloses a method for the simultaneous testing of the inhibition or induction/activation of major human drug-metabolizing cytochrome P450 isozymes, such as CYP3A4, CYP2D6, CYP2C9, CYP1A2, CYP2C19. CYP2A6, and CYP2C8, by potential drug candidates utilizing a cocktail of specific probe substrates for CYP450 isozymes is described. It also describes a method for determining the phenotype of a cell or a tissue sample by determining the CYP450 isozyme(s) present in the cell or tissue sample.

Chinese Patent No. CN102650620 relates to the preparation, detection method and application of a probe pharmaceutical composition for measuring the metabolic activity of cytochrome P450: the composition contains the specific probes of the main subtypes of CYP450, CYP1A2, CYP2C9, CYP2C19, CYP2D6, CYP2E1. Formulated as a “cocktail” probe drug solution, the probe drug composition is administered to animals or co-incubated with liver microsomes in vitro, and the concentration of each probe drug is measured to evaluate the CYP1A2. CYP2C9, CYP2C19, CYP2D6, CYP2E1 and CYP3A4 Metabolic activity; in the early stages of new drug development, high-throughput screening of drugs on the activity of cytochrome P450 subtypes to predict drug interactions.

Chinese Patent No. CN104928350 discloses a rapid screening method for comprehensively evaluating the inhibition effect of 9 human liver CYP450 metabolic enzymes in vitro by using 14 probe substrates and 16 probes. The invention mainly relates to monitoring the metabolic activity changes of nine human liver CYP450 enzymes by using an in vitro mixed probe incubation method and LC/MS/MS, and quickly and comprehensively evaluating the inhibitory effect of the test compound on metabolic enzymes.

Journal of Pharmacological and Toxicological Methods 58 (2008) 206-214: Michael Zientek et al; entitled “Development of an in vitro drug-drug interaction assay to simultaneously monitor five cytochrome P450 isoforms and performance assessment using drug library compounds” discloses inhibition of cytochrome P450 (CYP) as a principal mechanism for metabolism-based drug-drug interactions (DDIs). The article describes a robust, high-throughput CYP-mediated DDI assay using a cocktail of 5 clinically relevant probe substrates with quantification by liquid chromatography/tandem mass spectrometry (LC/MS-MS).

Biopharm Drug Dispos. 2019; 40:101-111; Hyun-Ji Kim et al; entitled “Screening of ten cytochrome P450 enzyme activities with 12 probe substrates in human liver microsomes using cocktail incubation and liquid chromatography-tandem mass spectrometry reports an in vitro HTS P450 cocktail inhibition assay containing 12 substrates for 10 P450 isoforms (three different substrates for CYP3A) developed using LC-MS/MS and validated using representative inhibitors of each P450 enzyme. The assay includes all seven P450 isoforms recommended by the US FDA as well as three additional isoforms, CYP2A6, CYP2E1 and CYP2J2.

A major hindrance in the implementation of cocktail approach is the turnover differences among probe substrates, leading to high protein concentrations, higher incubation times and probable interactions among probe substrates. Some studies have also reported subsets of cocktail incubations by separating the probe substrates into subgroups. Further, an analytical method with high sensitivity and selectivity is also desired. Till date several CYP inhibition by cocktail assays have been reported, but carried loopholes like using higher substrate concentrations, higher microsomal protein concentrations, higher organic content, and longer sample analysis time or unable to incorporate all substrates of CYP isoforms of interest into one cocktail. Therefore, there exists a need to obtain single incubation condition for cocktail of probe substrates of 8 major CYP isoforms to assess in vitro DDIs of novel drug candidates.

The inventors of the present invention have developed an assay to overcome the challenges discussed above using liquid chromatography coupled to tandem mass spectrometry (LC-MS/MS) method. The assay is designed to include all 7 CYP450 isoforms recommended for testing by USFDA as well as one additional isoform, CYP2A6, which is also important in the metabolism of xenobiotic compounds. Further, this high throughput method resulted in reduced turnaround time with reproducibility in data for evaluation of CYP inhibition potential of NCEs to drug discovery teams. In addition, the cocktail assay of the present invention reduces reagent and manpower requirement which eventually contributes to cost savings. This cocktail approach can be routinely used to provide speedy DDI data for discovery projects.

### SUMMARY OF THE INVENTION

In an aspect, the present invention relates to a cocktail method for simultaneous estimation of inhibition potential of xenobiotics for major CYP isoforms in human liver microsomes using 8 probe substrates.

In one aspect, the present invention relates to an improved substrate cocktail assay method wherein, the 8 major CYP consists of CYP3A4, CYP2C9, CYP2C19, CYP2D6, CYP1A2, CYP2B6, CYP2A6 and CYP2C8.

In one aspect, the present invention relates to a cocktail assay method, wherein the probe substrate consists of Testosterone specific to CYP isoform, CYP3A4; diclofenac specific to CYP isoform, CYP2C9; S-mephenytoin specific to CYP isoform, CYP2C19; bufuralol specific to CYP isoform, CYP2D6; phenacetin specific to CYP isoform, CYP1A2; bupropion specific to CYP isoform, CYP2B6; coumarine specific to CYP isoform, CYP2A6 and paclitaxel specific to CYP isoform, CYP2C8.

In another aspect, the present invention provides an improved substrate cocktail assay wherein the probe substrates selected at specified concentrations had minimal inhibition effect on the other isoform activities.

In yet another aspect, the present invention provides IC50 values in consistence with those obtained through the individual probe substrate assay.

In an aspect, the cocktail assay method is used to assess in vitro drug-drug interactions (DDIs) of novel drug candidates using liquid chromatography coupled to tandem mass spectrometry (LC-MS/MS) method.

### BRIEF DESCRIPTION OF THE DRAWINGS

Various aspects of the invention will be further explained with reference to embodiments shown in the drawings wherein:

FIGS. 1A to 1D depicts inhibition curves of known inhibitors obtained using single substrates and substrate cocktails.

FIGS. 2A to 2H depicts representative multiple reaction monitoring (MRM) chromatograms of CYP450-mediated metabolites in a human liver microsomal sample incubated with probe substrates: FIG. 2A—paclitaxel (4.63 min). FIG. 2B—bufuralol (2.26 min). FIG. 2C—bupropion (2.69 min), FIG. 2D—coumarin (2.53 min), FIG. 2E—diclofenac (4.41 min), FIG. 2F—S-mephenytoin (2.47 min). FIG. 2G—testosterone (3.40 min), FIG. 2H—phenacetin (1.61 min) and monitored in positive ion mode.

### DETAILED DESCRIPTION OF THE INVENTION

In general, the various terms used herein pertaining to the instantly presented invention are defined herein below:

It should be understood that the detailed description and specific examples, while indicating embodiments of the invention, are given by way of illustration only, since various changes and modifications within the spirit and scope of the invention will become apparent to those skilled in the art. One skilled in the art, based upon the definitions herein, may utilize the present invention to its fullest extent. The following specific embodiments are to be construed as merely illustrative, and not limitative of the remainder of the disclosure in any way whatsoever.

Unless otherwise defined, all the terms used herein, including the technical and scientific terms, have the meaning as that generally understood by one of ordinary skill in the art to which the present invention relates.

In an embodiment, the present invention relates to an improved substrate cocktail assay method for simultaneous estimation of inhibition potential of xenobiotics for major CYP isoforms in human liver microsomes using 8 probe substrates.

In another embodiment, the present invention relates to a cocktail assay method, wherein the 8 major CYP consists of CYP3A4, CYP2C9, CYP2C19, CYP2D6, CYP1A2, CYP2B6, CYP2A6 and CYP2C8.

In an embodiment, the present invention relates to a cocktail assay method, wherein the probe substrates for each CYP450 enzyme is selected based on the minimum or no interaction of CYP450 enzyme with other probe substrates in cocktail.

In another embodiment, the present invention relates to a cocktail assay method, wherein the microsomal protein concentration is low.

In another embodiment, the present invention relates to a cocktail assay method, wherein the probe substrate consists of Testosterone specific to CYP isoform, CYP3A4; diclofenac specific to CYP isoform, CYP2C9; S-mephenytoin specific to CYP isoform, CYP2C19; bufuralol specific to CYP isoform, CYP2D6: phenacetin specific to CYP isoform, CYP1A2; bupropion specific to CYP isoform, CYP2B6; coumarine specific to CYP isoform, CYP2A6 and paclitaxel specific to CYP isoform, CYP2C8.

In an embodiment, the probe substrate metabolites selected for CYP1A2, CYP2B6 and CYP2A6 are Phenacetin O-deethylation (paracetamol), bupropion-hydroxylation and coumarin-7-hydroxyation metabolites respectively, which are based on superior specificity/availability compared to other USFDA-recommended substrates.

In another embodiment, the probe substrates metabolites selected for CYP2D6 and CYP2C9 are bufuralol hydroxylation and diclofenac hydroxylation respectively.

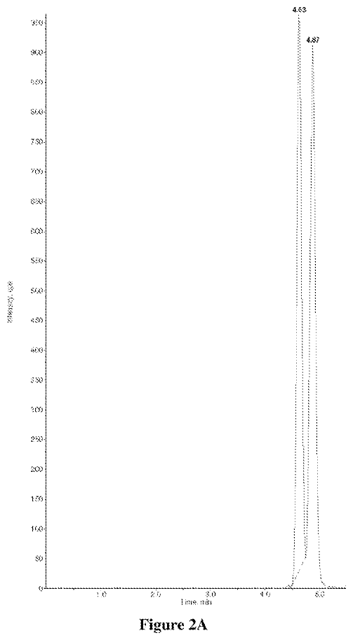

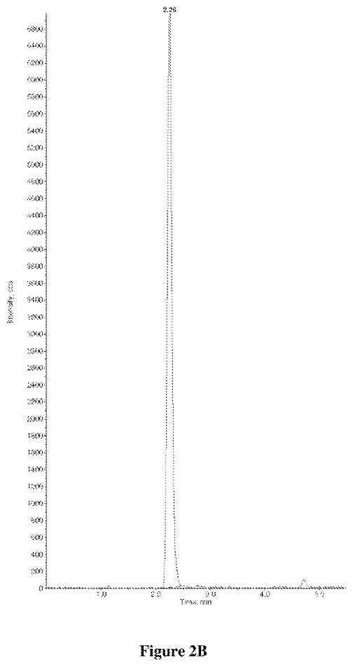

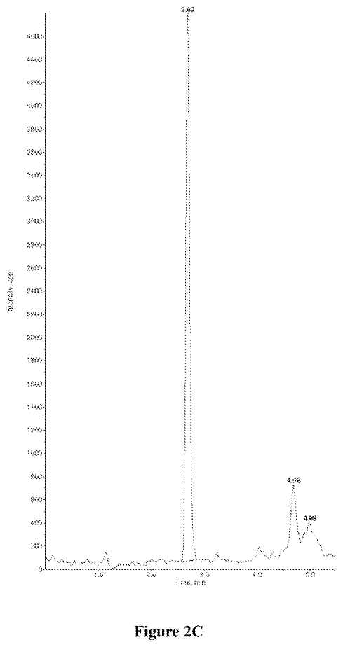

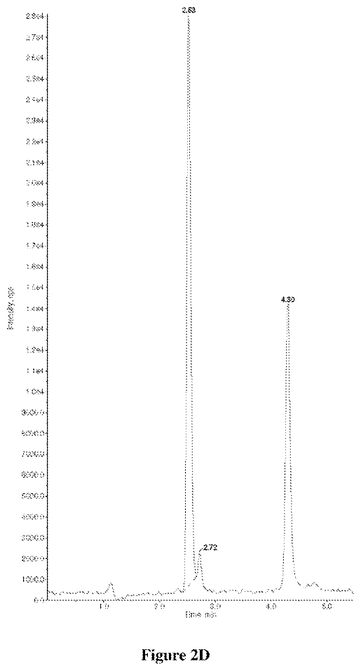

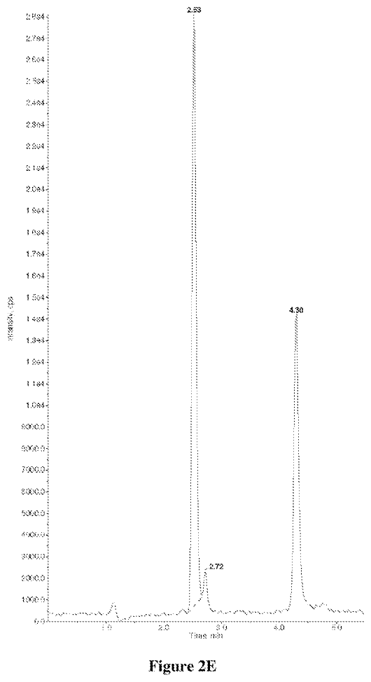

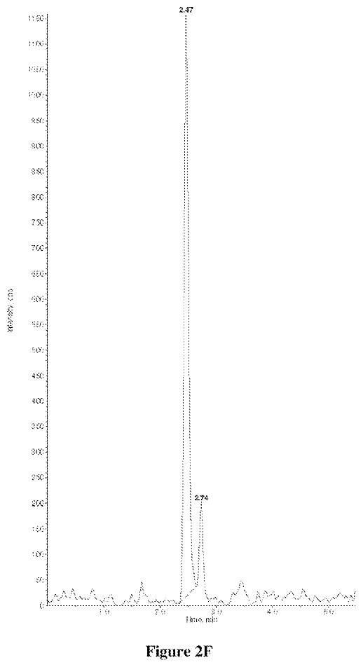

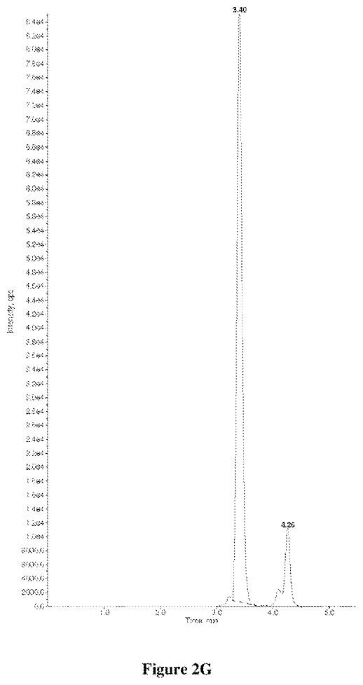

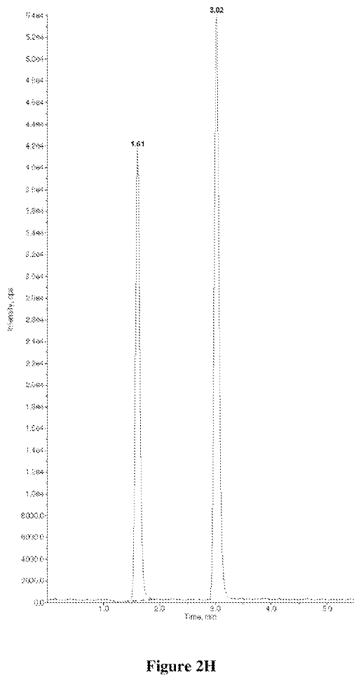

In another embodiment the probe substrate metabolite selected for CYP2C19 is (S)-mephenytoin, owing to enough sensitivity of LC-MS/MS detection (FIGS. 2A-2H).

In yet another embodiment, the probe substrate metabolite selected for CYP3A4 is testosterone 6β-hydroxylation as recommended by USFDA.

In further embodiment, the probe substrate metabolite selected for CYP2C8 is paclitaxel hydroxylation owing to its high sensitivity.

In an embodiment, the present invention provide a cocktail assay method wherein, the positive control inhibitors are selected from the group consisting of ketoconazole, sulphaphenazole, benzyl-nirvanol, quinidine, α-naphthoflavone, ticlopidine, methoxsalen and montelukast.

In yet another embodiment, the present invention provides an improved substrate cocktail assay wherein the probe substrates selected at specified concentrations had minimal inhibition effect on the other isoform activities.

In one embodiment, the present invention provides an assay wherein, the final probe substrate concentrations in the cocktail assay was kept less than reported Michaelis-Menten constant (Km) of the probe substrate for the corresponding cytochrome P450 enzymes in order to minimize probable interactions between probe substrates as indicated in Table 1.

TABLE 1
Km values and concentrations of CYP-specific
probe substrates used in the cocktail assays

| | | | Concentra- | |
|---|---|---|---|---|
| Iso- | | | tion | Km |
| form | Substrate | Metabolite | (μM) | (μM) |
| 3A4 | Testosterone | hydroxyl- | 50 | 48.45 ± 3.75 |
| | | testosterone | | |
| 2D6 | Bufuralol | hydroxyl-bufuralol | 3 | 7 ± 3 |
| 1A2 | Phenacetin | Paracetamol | 30 | 39.0 ± 1.9 |
| 2C9 | Diclofenac | hydroxyl-diclofenac | 5 | 22 ± 11 |
| 2C19 | S- | hydroxyl- | 50 | 54.85 ± 6.45 |
| | mephenytoin | mephenytoin | | |
| 2B6 | Bupropion | hydroxyl-bupropion | 3 | 103.45 ± 7.65 |
| 2A6 | Coumarin | hydroxyl-coumarine | 1 | 1.17 ± 0.118 |
| 2C8 | Paclitaxel | 6-alpha hydroxyl- | 5 | 11.5 ± 7.5 |
| | | paclitaxel | | |

The above table shows that lowering substrate concentration was the most efficient strategy, but detection limit of LCMS/MS was also considered.
The probe substrate for 2B6 (bupropion) has been reported to strongly inhibit activities of CYP2D6 and 2C19 at 12 μM. To minimize this interaction, bupropion concentration in the present cocktail assay was reduced to 3 μM along with replacing dextromethorphan as substrate with bufuralol for CYP2D6 and using more potent inhibitor than ticlopidine i.e. benzyl-nirvanol for CYP2C19. The obtained IC50 values reduced from 0.84 to 0.036 (23-folds) and 5.81 to 0.289 (20-folds) for CYP 2D6 and CYP 2C19 respectively, indicating prominence and suitability of the presently developed cocktail assay.
The concentration of bupropion was taken 30 times lower than Km and to diminish the interaction, concentration of bufuralol (CYP 2D6 substrate) was also reduced from 5 μM, to 3 μM.
Phenacetin (1A2 substrate) has been reported to weakly inhibit CYP3A4 and 2B6. Reducing concentration of phenacetin from 100 μM to 30 μM, improved IC50 by 2-fold for 3A4 and 10-fold for 2B6.
Lowest possible concentrations of coumarin, diclofenac and paclitaxel have been used to avoid any probable interactions.

The metabolite formation was then investigated for the incubation time of 20 min as it was the minimum required time to ensure sufficient metabolite formation for the low-turnover substrates like S-mephenytoin and diclofenac.

In further embodiment, the present invention provides IC50 values in consistence with those obtained through the individual probe substrate assay as indicated in Table 2.

TABLE 2
Comparison of IC50 values of known inhibitors obtained using the single
substrate (individual) approach, cocktail approach and literature values

| | | | | | Single Substrate | | Cocktail | |
|---|---|---|---|---|---|---|---|---|
| | | | Substrate | Substrate | (n = 4) | | (n = 4) | |
| | | | Conc. (μM) | Conc. (μM) | IC50 | Literature | IC50 | Literature |
| Isoform | Inhibitor | Substrate | Individual | Cocktail | (μM) | IC50 | (μM) | IC50 |
| 3A4 | Ketoconazole | Testosterone | 70 | 50 | 0.101 ± | 0.059 ± | 0.024 ± | 0.016 ± |
| | | | | | 0.019 | 0.005 | 0.008 | 0.0027 |
| 2D6 | Quinidine | Bufuralol | 5 | 3 | 0.042 ± | 0.106 ± | 0.036 ± | 0.013 |
| | | | | | 0.018 | 0.034 | 0.017 | |
| 1A2 | α-napthoflavone | Phenacetin | 30 | 30 | 0.071 ± | 0.0318 ± | 0.026 ± | 0.025 ± |
| | | | | | 0.020 | 0.002 | 0.007 | 0.0001 |
| 2C9 | Sulfaphenazole | Diclofenac | 20 | 5 | 1.057 ± | 0.973 ± | 0.492 ± | 0.6095 ± |
| | | | | | 0.046 | 0.139 | 0.122 | 0.03 |
| 2C19 | Benzyl nirvanol | S-mephenytoin | 50 | 50 | 0.346 ± | 0.412 ± | 0.289 ± | 0.23 ± |
| | | | | | 0.072 | 0.0825 | 0.059 | 0.05 |
| 2B6 | Ticlopidine | Bupropion | 6 | 3 | 0.249 ± | 0.325 | 0.105 ± | 0.625 ± |
| | | | | | 0.045 | | 0.017 | 0.05 |
| 2A6 | Methoxsalen | Coumarin | 1 | 1 | 0.315 ± | 0.46 ± | 0.2 ± | 0.5 ± |
| | | | | | 0.021 | 0.03 | 0.054 | 0.05 |
| 2C8 | Montelukast | Paclitaxel | 5 | 5 | 0.521 ± | — | 0.258 ± | — |
| | | | | | 0.064 | | 0.15 | |

From the above table it is evident that the ratios of the IC50 values obtained from individual upon cocktail approaches were found up to 4.2 which clearly demonstrated improvement in achieving lower IC50 values in cocktail assay. The higher IC50 values in individual assay would be due to longer incubation time and high protein concentration leading to non-specific binding of substrates (if any). Further, range of obtained IC50 values in cocktail assay was much narrower, such as 0.0240±0.008 for ketoconazole, 0.026±0.007 for α-naphthoflavone and 0.10.5±0.017 for ticlopidine in comparison to the cocktail assays reported in literature. Additionally, the obtained IC50 values were in line with previously reported data (table 1).

The data suggests that cocktail assay can be used to accurately determine IC50 values of novel drug candidates against CYP450 isoform-mediated metabolism instead of individual substrate incubations, which would save a remarkable amount of time and resources in estimating DDI potential of NCEs.

The invention is further illustrated by the following examples which are provided to be exemplary of the invention, and do not limit the scope of the invention. While the present invention has been described in terms of its specific embodiments, certain modifications and equivalents will be apparent to those skilled in the art and are intended to be included within the scope of the present invention.

### EXAMPLES

### Materials & Methods:

Testosterone, diclofenac, phenacetin, bupropion, coumarine, ketoconazole, sulphaphenazole, quinidine, α-naphthoflavone, ticlopidine, methoxsalen, propranolol, potassium phosphate monobasic, potassium phosphate dibasic and dimethyl sulfoxide (DMSO) were purchased from Sigma-Aldrich (St. Louis, Mo., USA). β-nicotinamide adenine dinucleotide phosphate (NADPH) was procured from Sisco Research Laboratories (Mumbai, India). S-mephenytoin, bufuralol hydrochloride, paclitaxel, benzyl-nirvanol and montelukast were obtained from Toronto Research Chemicals (Toronto, Canada). Pooled human liver microsomes (200 donors) were purchased from Sekisui XenoTech (KS, USA). Formic acid, LC/MS-grade acetonitrile and methanol were purchased from Merck (Darmstadt, Germany). Water was purified using Milli-Q PLUS PF (Billerica, Mass. USA) water purification system.

The microsomal incubations using human liver microsomes (HLM; 0.1 mg/mL) were conducted with potassium phosphate buffer (0.1 M, pH 7.4) having 5 mM magnesium chloride, 1.5 mM NADPH, a single substrate or cocktail of 8 probe substrates [testosterone (3A4; 50 mM in ACN), diclofenac (2C9; 10 mM in DMSO), S-mephenytoin (2C19; 50 mM in Methanol), bufuralol hydrochloride (2D6; 10 mM in DMSO), phenacetin (1A2; 50 mM in ACN), bupropion (2B6; 10 mM in Methanol), coumarine (2A6; 5 mM in Methanol) and paclitaxel (2C8; 25 mM in DMSO)] and respective positive control inhibitors. The substrates were used at concentrations approximately near or below their respective Km values to minimize any unwanted interactions among P450 probe substrates (table 1).

The known CYP inhibitors as recommended by USFDA for both individual as well as cocktail assays were used at 6 different concentrations as follows: 0.0016-5 μM for ketoconazole (3A4), 0.024-10 μM for sulphaphenazole (2C9), 0.012-5 μM for benzyl-nirvanol (2C19), 0.0016-5 μM for quinidine (2D6), 0.0016-5 μM for α-naphthoflavone (1A2), 0.012-5 μM for ticlopidine (2B6), 0.024-10 μM methoxsalen for (2A6), and 0.024-10 μM for montelukast (2C8).

At start of the experiment, various concentrations of positive control inhibitors as described to above were prepared in assay buffer. For 100 μL of final reaction system, 0.5 μL of HLM (20 mg/ml), 50 μL of reference compound (from each concentration) and 19.5 μl of buffer were added. For pre-incubation, reaction plates were kept in shaker for 5 min at 37° C., 4(0) rpm. After pre-incubation, 20 μl of substrate mixture and 10 μL of cofactor (NADPH −15 mM) were added to the wells. The reaction was then allowed for 20 min incubation at 37° C. After completion of the incubation period, reaction was terminated with 200 μL of chilled methanol:water (80:20; % v/v) containing internal standard (Propranolol). The samples were then centrifuged (4000 rpm: 4° C.) for 20 min and supernatants were analysed using LCMS/MS. After completion of incubation with probe substrates and quantitative analysis of metabolites. IC50 values were calculated. The total organic solvent concentration of 0.740% was achieved uniformly across all the reactions in the assay (DMSO concentration: 0.430%+other organic solvent: 0.310%). The organic content was kept less than 1% to avoid inhibition/activation impact on CYP isoforms.

The individual approach differed from cocktail in incubation time i.e. 45 min for 3A4 and 2C19; 30 min for 1A2, 2C9, 2B6 and 2C8; 20 min for 2A6 and 10 min for 2D6. The substrate concentrations were kept similar except for 3A4, 2D6, 2C9 and 2B6, where higher substrate concentrations were used as stated in table 2. Further, the microsomal protein concentration was kept 0.2 mg/mL for individual incubations.

### Data Analysis:

Data was expressed as mean t SD unless otherwise indicated. The data normalization was done with respect to internal standard and % inhibition was calculated with respect to DMSO control using Microsoft Excel (Seattle, Wash.). The IC50 values were calculated using Graph Pad Prism software (version 5.03; CA, USA).

### LC-MS/MS Analysis:

All metabolites and the internal standard were analysed in a single run using a SCIEX LCMS/MS API 4000 tandem mass spectrometer coupled with a Nexera high-performance liquid chromatography system (Shimadzu, Kyoto. Japan) equipped with an electrospray ionization interface. The 8 metabolites and internal standard were separated on a X-Bridge-C18 column (100×4.6 mm, 5 μm, 100 Å; Waters). The mobile phase consisted of 2 mM ammonium acetate with 0.1% formic acid in water (A) and Methanol (B), and was set as 40% B (0-0.5 min). 40%→60% B (0.5-1.2 min), 60% B (1.2-2.2 min), 60%→75% B (2.2-2.5 min), 75% B (2.5-4.6 min), 75%→40% B (4.6-4.8 min), and 40% B (4.8-5 min). The total run time was 5.5 min, and the flow rate was 0.8 ml/min. Electrospray ionization was performed in positive-ion mode at 5500 V. The optimum operating conditions were determined as follows: Source temperature, 350° C.; Ion Spray Voltage, 5500 V; Ion Source Gas1, 40 psi; Ion Source Gas2, 60 psi; Curtain gas 20 and Collision gas 8. Quantitation was conducted in multiple reaction monitoring (MRM) modes with the precursor-to-product ion transition for each metabolite (Table 4). The data acquisition was ascertained by Analyst™ (version 1.6.2; Applied Biosystems. Toronto. Canada).

### Results:

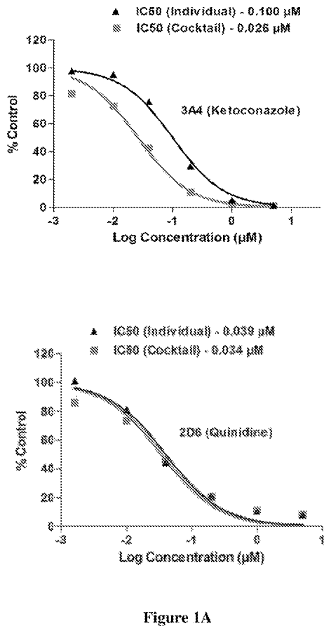

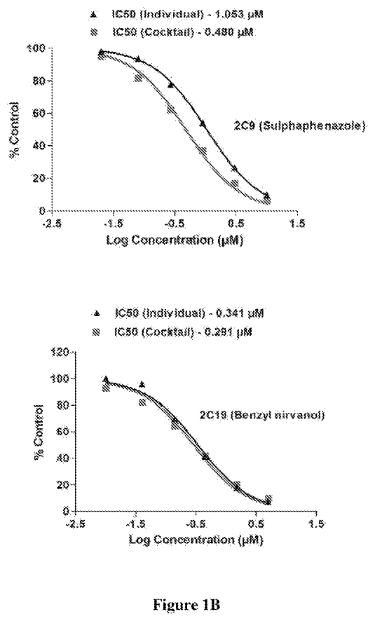

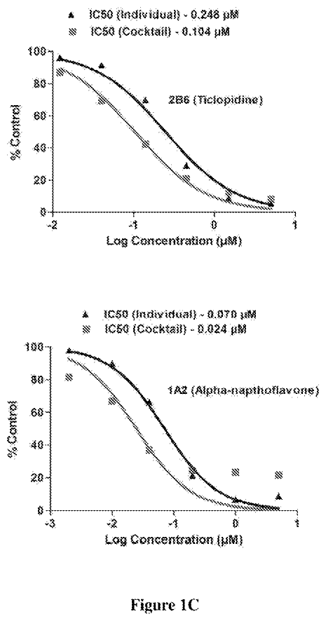

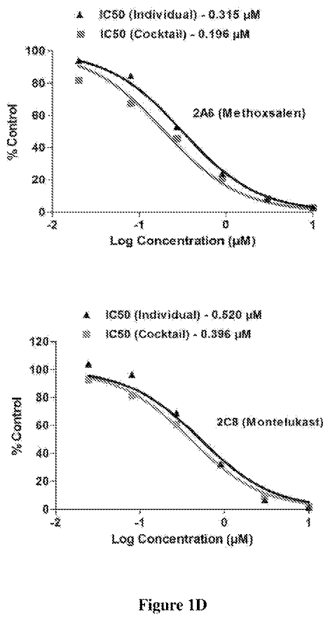

The probe substrates specific to CYP isoforms were selected based on USFDA recommendation, specificity of the enzymatic reaction and sensitivity of analytical detection. The final concentration of each substrate used in the assay was based on literature reported Km value (table 1). An incubation time of 20 min was selected based on formation of all 8 metabolites during this period in sufficient amount, as well as detectable peak intensity of low sensitivity analytes like hydroxyl-mephenytoin. The microsomal protein concentration was fixed to 0.1 mg/mL to avoid any non-specific binding of substrates with the microsomal proteins. The IC50 values obtained using the cocktail approach were compared and found to be in agreement with those obtained using individual substrates and the values reported in the literature for validation of the cocktail assay (Table 2). Inhibition curves of CYP-specific inhibitors using single substrate/cocktail approach with respective IC50 values are depicted in FIGS. 1A-1D.

### Advantages of the Present Invention:

Improved and reduced substrate concentration in comparison with the existing literatures (cocktail of 8 substrates atone go).
Developed in lower microsomal protein to avoid any non-specific binding of substrates with the microsomal proteins.
Reduced total organic content (<0.75%) and DMSO conc. restricted to 0.43%). This has enabled to overcome the impact on certain key isoforms.

TABLE 3
Results obtained from the cocktail substrate assay of the present invention as demonstrated herein above.
(Cocktail substrate assay)

| | | | | Substrate | | Microsomal | Incubation | Run | Organic |
|---|---|---|---|---|---|---|---|---|---|
| | | | | Concentration | | conc | time | time | content |
| Isoform | Substrate | Metabolite | Inhibitor | (μM) | IC50 | (mg/ml) | (min) | (min) | (%) |
| 3A4 | Testosterone | hydroxyl- | Ketoconazole | 50 | 0.024 ± | 0.1 | 20 | 5 | 0.74 |
| | | testosterone | | | 0.008 | | | | |
| 2D6 | Bufuralol | hydroxyl- | Quinidine | 3 | 0.036 ± | | | | |
| | | bufuralol | | | 0.017 | | | | |
| 1A2 | Phenacetin | Paracetamol | a-napthoflavone | 30 | 0.026 ± | | | | |
| | | | | | 0.007 | | | | |
| 2C9 | Diclofenac | hydroxyl- | Sulfaphenazole | 5 | 0.492 ± | | | | |
| | | diclofenac | | | 0.122 | | | | |
| 2C19 | S-mephenytoin | hydroxyl- | Benzyl | 50 | 0.289 ± | | | | |
| | | mephenytoin | nirvanol | | 0.059 | | | | |
| 2B6 | Bupropion | hydroxyl- | Ticlopidine | 3 | 0.105 ± | | | | |
| | | bupropion | | | 0.017 | | | | |
| 2A6 | Coumarin | hydroxyl- | Methoxsalen | 1 | 0.2 ± | | | | |
| | | coumarine | | | 0.054 | | | | |
| 2C8 | Paclitaxel | 6-alpha | Motelukast | 5 | 0.258 ± | | | | |
| | | hydroxyl- | | | 0.15 | | | | |
| | | paclitaxel | | | | | | | |

The MRM transitions for all 8 metabolites and Internal Standard (IS) were selected based on the most abundant fragment ions and recorded in table 4. All metabolites and IS were monitored in positive-ion mode. The optimized compound parameters and LC column retention times for the respective metabolites were also reported in table 4. Typical MRM mode chromatograms of all metabolites detected during LC-MS/MS run shown in FIGS. 2A-2H. Typical maximum signal intensities are listed on the MRM chromatogram with the highest intensity being 5.5×105 for hydroxyl-testosterone and the lowest intensity being 0.6×104 for hydroxyl-mephenytoin. Note that all metabolites eluted within 5.5 min. This run time was short enough to allow high throughput screening of novel NCEs. It was assured that any kind of interfering impurities are not eluting out at the retention time of metabolites or IS.

TABLE 4
Optimized multiple reaction monitoring parameters
for analyte and internal standard.

| | | | | | | | | RT |
|---|---|---|---|---|---|---|---|---|
| Isoform | Metabolite | Q1 | Q3 | DP | EP | CE | CXP | (min) |
| 3A4 | hydroxyl- | 305.124 | 269.1 | 100 | 10 | 23 | 19 | 3.40 |
| | testosterone | | | | | | | |
| 2D6 | hydroxyl- | 278.1 | 186.0 | 60 | 10 | 27 | 10 | 2.26 |
| | bufuralol | | | | | | | |
| 1A2 | Paracetamol | 152.1 | 110.0 | 69 | 10 | 22 | 10 | 1.61 |
| 2C9 | hydroxyl- | 312.1 | 265.9 | 48 | 10 | 20 | 21 | 4.41 |
| | diclofenac | | | | | | | |
| 2C19 | hydroxyl- | 235.1 | 150.1 | 61 | 10 | 25 | 11 | 2.47 |
| | mephenytoin | | | | | | | |
| 2B6 | hydroxyl- | 256.2 | 238.1 | 80 | 10 | 18 | 11 | 2.69 |
| | bupropion | | | | | | | |
| 2A6 | hydroxyl- | 163.1 | 107.1 | 70 | 10 | 30 | 20 | 2.53 |
| | coumarine | | | | | | | |
| 2C8 | 6-alpha | 870.5 | 286.3 | 63 | 8 | 23 | 7 | 4.63 |
| | hydroxyl - | | | | | | | |
| | paclitaxel | | | | | | | |
| Internal | Propranolol | 260.2 | 116.1 | 94 | 10 | 26 | 7 | 3.04 |
| standard | | | | | | | | |

Q1: Precursor ion;
Q3: Product ion;
DP: Declustering Potential;
EP: Entrance Potential;
CE: Collision Energy;
CXP: Collision Cell Exit Potential;
RT: Retention Time

We claim:

1. An improved substrate cocktail assay method for simultaneous estimation of inhibition of potential of xenobiotics for major CYP isoforms in human liver microsomes using 8 probe substrates.

2. The cocktail assay method as claimed in claim 1, wherein the 8 major CYP consists of CYP3A4, CYP2C9, CYP2C19, CYP2D6, CYP1A2, CYP2B6, CYP2A6 and CYP2C8.

3. The cocktail assay method as claimed in claim 1, wherein the probe substrates for each CYP450 enzyme is selected based on the minimum or no interaction of CYP450 enzyme with other probe substrates in cocktail.

4. The cocktail assay method as claimed in claim 1, wherein the microsomal protein concentration is low.

5. The cocktail assay method as claimed in claim 1, wherein the probe substrate consists of Testosterone specific to CYP isoform, CYP3A4; diclofenac specific to CYP isoform, CYP2C9; S-mephenytoin specific to CYP isoform, CYP2C19; bufuralol specific to CYP isoform, CYP2D6; phenacetin specific to CYP isoform, CYP1A2; bupropion specific to CYP isoform, CYP2B6; coumarine specific to CYP isoform, CYP2A6 and paclitaxel specific to CYP isoform, CYP2C8.

6. The cocktail assay method as claimed in claim 1, wherein the IC50 values are in consistence with those obtained through the individual probe substrate assay.

7. The cocktail assay method as claimed in claim 1, wherein the final probe substrate concentrations in the cocktail assay was kept less than reported Michaelis-Menten constant (Km) of the probe substrate for the corresponding cytochrome P450 enzymes in order to minimize probable interactions between probe substrates.

8. The cocktail assay method as claimed in claim 1, wherein said method is developed in lower microsomal protein having concentration fixed to 0.1 mg/mL to avoid non-specific binding of substrates with the microsomal proteins and contains reduced total organic content, <0.75% and DMSO concentration restricted to 0.43%.

9. The cocktail assay method as claimed in claim 1, wherein said method is used to assess in vitro drug-drug interactions of novel drug candidates using liquid chromatography coupled to tandem mass spectrometry method.
